SEQUENCE LISTING (21 sequences)

SEQ ID NO: 1 - RNA, 20 nt, Artificial Sequence
  Description of Artificial Sequence Synthetic oligonucleotide
cungaacaccggcaaaugca

SEQ ID NO: 2 - RNA, 21 nt, Artificial Sequence
  Description of Artificial Sequence Synthetic oligonucleotide
ncuuguggccguuuacgucgc

SEQ ID NO: 3 - RNA, 19 nt, Artificial Sequence
  Description of Artificial Sequence Synthetic oligonucleotide
ncuuguggccguuuacguc

SEQ ID NO: 4 - RNA, 18 nt, Artificial Sequence
  Description of Artificial Sequence Synthetic oligonucleotide
ugcauuugccgguguuca

SEQ ID NO: 5 - RNA, 21 nt, Artificial Sequence
  Description of Artificial Sequence Synthetic oligonucleotide
gacguaaacggccacaaguuc

SEQ ID NO: 6 - RNA, 19 nt, Artificial Sequence
  Description of Artificial Sequence Synthetic oligonucleotide
gacguaaacggccacaagu

SEQ ID NO: 7 - protein, 4 aa, Artificial Sequence
  Description of Artificial Sequence Synthetic peptide
GKKK

SEQ ID NO: 8 - protein, 5 aa, Artificial Sequence
  Description of Artificial Sequence Synthetic peptide
AGKKK

SEQ ID NO: 9 - protein, 6 aa, Artificial Sequence
  Description of Artificial Sequence Synthetic peptide
AGGKKK

SEQ ID NO: 10 - protein, 5 aa, Artificial Sequence
  Description of Artificial Sequence Synthetic peptide
GKKKK

SEQ ID NO: 11 - protein, 6 aa, Artificial Sequence
  Description of Artificial Sequence Synthetic peptide
GGKKKK

SEQ ID NO: 12 - protein, 6 aa, Artificial Sequence
  Description of Artificial Sequence Synthetic peptide
AGKKKK

SEQ ID NO: 13 - protein, 4 aa, Artificial Sequence
  Description of Artificial Sequence Synthetic peptide
GRRR

SEQ ID NO: 14 - protein, 8 aa, Artificial Sequence
  Description of Artificial Sequence Synthetic peptide
GRRRGKKK

SEQ ID NO: 15 - protein, 10 aa, Artificial Sequence
  Description of Artificial Sequence Synthetic peptide
GGKKKGKKKK

SEQ ID NO: 16 - protein, 8 aa, Artificial Sequence
  Description of Artificial Sequence Synthetic peptide
GGKKKKKK

SEQ ID NO: 17 - protein, 12 aa, Artificial Sequence
  Description of Artificial Sequence Synthetic peptide
GGKKKKKKKGRG

SEQ ID NO: 18 - protein, 10 aa, Artificial Sequence
  Description of Artificial Sequence Synthetic peptide
GGKKKKKKKG

SEQ ID NO: 19 - protein, 10 aa, Artificial Sequence
  Description of Artificial Sequence Synthetic peptide
GGKKKGKKKG

SEQ ID NO: 20 - protein, 8 aa, Artificial Sequence
  Description of Artificial Sequence Synthetic peptide
KKKKGKKK

SEQ ID NO: 21 - protein, 8 aa, Artificial Sequence
  Description of Artificial Sequence Synthetic peptide
KKKGKKKK

(19) United States
(12) Patent Application Publication
(10) Pub. No.: US 2010/0197041 A1
(43) Pub. Date: Aug. 5, 2010

(54) NUCLEIC ACID BINDING ASSAYS

(76) Inventor: Thomas Hermann, Cardiff by the Sea, CA (US)

Correspondence Address: FOLEY & LARDNER LLP, P.O. BOX 80278, SAN DIEGO, CA, 92138-0278, US

(21) Appl. No.: 12/695,089

(22) Filed: Jan. 27, 2010

Related U.S. Application Data
(60) Provisional application No. 61/148,832, filed on Jan. 30, 2009.

(51) Int. Cl. G01N 33/566

(52) U.S. Cl. 436/501

(57) ABSTRACT

This invention relates to methods for screening compounds for the ability to interact with a nucleic acid target, assay kits useful thereof and compositions regarding same. In a particular aspect, the invention relates to specific binding assays employing fluorescent label(s). The methods involve assessing the conformation of nuclei acid targets in the presence and absence of test compounds, and identifying as a ligand any test ligand that causes a measurable conformation change in nuclei acid targets. The effect of compounds on target nuclei acids conformation is assessed by measuring the fluorescence changes of a fluorescently label(s) attached hereto.

### RELATED APPLICATION

This application claims benefit of priority from U.S. provisional application Ser. No. 61/148,832 filed Jan. 30, 2009 entitled “Nucleic Acid Binding Assays” which is incorporated by reference herein in its entirety.

### SEQUENCE LISTING

The present application contains a Sequence Listing which has been submitted via EFS-Web and is hereby incorporated by reference in its entirety. Said ASCII copy, created on March 4, is named 093369US.txt, and is 5,255 bytes in size.

### TECHNICAL FIELD

This invention relates to methods for screening compounds for the ability to interact with nucleic acids and assay kits useful therefor. In a particular aspect, the invention relates to specific binding assays utilizing fluorescence.

### BACKGROUND

An electrophoretic mobility shift assay (EMSA), also referred as a gel shift assay, is a common technique used to study protein-polynucleotide interactions. This procedure can determine if a protein or mixture of proteins is capable of binding to a given DNA or RNA sequence. In the same manner, gel shift assay was developed to determine if a complex is formed between the non-protein macromolecules and siRNA, providing a tool for screening molecules that bind siRNA. (see e.g. Gamer, M. M. et al., Nucleic Acids Res. 1981, 9:3047-306©; Fried, M. et al. Nucleic Acids Res., 1981, 9:6505-6525). However, gel shift assays could not provide quantitative measurements regarding binding affinity or provide information regarding determination of the binding site.

Fluorescence labeling of nucleic acids (DNA and RNA) has been used for a long time to monitor strand hybridization, folding and ligand binding, including the binding of proteins, peptides and small molecules. For example, the use of fluorescent nucleobase analogs, e.g. 2-aminopurine (2AP), in place of a nucleobase of choice, for the study of conformational or structural changes in biopolymers has been reported. (Ward, et al., J Biol Chem. 1969, 244(5):1228-37). 2AP, positioned in the middle of a DNA duplex, was used for labeling DNA (Patel, et al., Eur J Biochem. 1992, 203(3):361-6) and 2AP, positioned internal to a folded RNA sequence, was used for labeling RNA (Lacourciere, et al., Biochemistry. 2000,39(19):5630-41).

2AP was also reported to be used to monitor binding of small molecule ligands that alter the conformation of the fluorescent labeled RNA (Kaul et al., J Am Chem Soc. 2004 126(11):3447-53; Shandrick, et al., Angew Chem Int Ed Engl. 2004, 43(24):3177-82; Bradrick, et al., RNA. 2004, 10(9):1459-68). Besides 2AP, pteridine nucleoside analogs such as 3-methyl isoxanthopterin (3MI) and 6-methyl isoxanthopterin (6MI), are also reported to be suitable for the study of conformational or structural changes in nucleic acids (Hawkins M. E. Cell Biochem Biophys. 2001, 34(2):257-81) or for the study of ligand binding to RNA (Parsons, et al., Tetrahedron 2007, 63, 3548-52). These assays detect a quenching effect on fluorescence emitted by the fluorescent labeled polynucleotide resulting from binding.

Alternatively, fluorescent labels, e.g. pyrene, may be attached via linkers to nucleobases for monitoring nucleic acid-protein interactions that would result an increase of fluorescence upon binding (Preuss, et al., J Mol Biol. 1997, 273(3):600-13). The use of pyrene-labeled RNA, positioned internal to a folded RNA sequence, to monitor binding of small molecule ligands that alter the conformation of the fluorescent labeled RNA was also reported (Blount, et al., Nucleic Acids Res. 2003, 31(19):5490-500).

### SUMMARY OF INVENTION

In accordance with the present invention, there are provided methods for screening compounds for the ability to interact with nucleic acid targets via measuring the fluorescence of fluorescent label(s) at one or both termini of the nucleic acid targets. Also provided are assay kits useful therefor. Compositions of novel nucleic acid targets with fluorescent label(s) are also provided.

In one aspect, the invention provides methods fur screening compounds for the ability to interact with a nucleic acid target, comprising:
contacting a nucleic acid target with a test compound; and
measuring the fluorescence of the nucleic acid target, wherein the nucleic acid target has been modified by the incorporation of fluorescent label(s) at one or both termini of said nucleic acid target, whereby the change of fluorescence is indicative of the interaction of said compounds with said nucleic acid target.

In another aspect, the invention provides methods for screening compounds for the ability to interact with a nucleic acid target comprising measuring the fluorescence of the nucleic acid target after said nucleic acid target has been contacted with a test compound, wherein said nucleic acid target has been modified by the incorporation of fluorescent label(s) at one or both termini thereof.

In yet another aspect, the invention provides compositions comprising nucleic acid(s) having fluorescent label(s) attached at one or both termini thereof via a linker wherein the linker is a linear chain of C₂-C₂₀ alkyl, or —(X(CH2)ₘ)ₙ,— wherein X is independently O, S, NH, C═O, O—C—O or NHC═O, m=1-5 and n=1-7.

In yet another aspect, the invention provides assay kits for screening for compounds that bind a nucleic acid target at one or both termini thereof, comprising a nucleic acid modified by the incorporation of fluorescent label(s) at one or both termini thereof.

### BRIEF DESCRIPTION OF THE DRAWINGS

FIGS. 1A-1F represent examples of isotherms for a binding assay according to the invention utilizing pyrene labeled RNA.

FIGS. 2A and 2B represent examples of isotherms for a binding assay according to the invention utilizing 2AP labeled RNA.

FIG. 3 illustrates the quenching of fluorescence as a result of interaction of a target nucleic acid with a binding compound according to the present invention.

FIG. 4 illustrates the increase in fluorescence (relative to the stacked conformation) due to release of the fluorescent label(s) from the stacking conformation (as a result of the interaction of a test compound with a nucleic acid target at the labeled terminus of the nucleic acid target).

### DETAILED DESCRIPTION OF INVENTION

The present invention is directed to assays utilizing nucleic acid having fluorescent label(s) on a terminal base pair in a nucleic acid duplex (with or without linker) to monitor the binding of a compound that interacts with the terminal base pair wherein binding results in a change of fluorescence.

In accordance with the present invention, there are provided methods for screening compounds for the ability to interact with a nucleic acid target, comprising:

contacting a nucleic acid target with a test compound; and

measuring the fluorescence of the nucleic acid target, wherein the nucleic acid target has been modified by the incorporation of fluorescent label(s) at one or both termini of said nucleic acid target, whereby the change of fluorescence is indicative of the interaction of said compounds with to said nucleic acid target.

Fluorescent label(s) contemplated for use herein may comprise fluorescent nucleobase analogue(s), such as 2-aminopurine (2AP), that replace nucleobase(s) at one or both of the terminus nucleotide(s) of the target nucleic acid. In the absence of a test compound that interacts with the nucleic acid target, the fluorescent nucleobase analogue, upon excitation with light of the appropriate wavelength, will emit a first level of fluorescence (“high fluorescence”). Upon association of a test compound with the labeled terminus of the nucleic acid target, fluorescence is reduced (“quenched”) as a result of interaction of the target nucleic acid with the binding compound (see FIG. 3). The degree of fluorescence decrease correlates with the binding affinity and concentration of the binding ligand. Fore example, the higher the affinity, the greater the degree of quenching. Similarly, within certain concentration ranges, the higher the concentration, the greater the degree of quenching. Therefore, measurement of the fluorescence signal as a function of the test compound concentration will allow the quantitative determination of the binding affinity.

On the other hand, fluorescent label(s) such as pyrene may be attached to a nucleotide in proximity of the nucleic acid target terminus via a linker. The potential site of attaching linker to the nucleic acid target may include a nucleotide of the terminal base pair, the penultimate base pair or the overhang. The linker may be attached to the base, the phosphate or the sugar of the nucleotide, e.g., at C2′, C3′, C4′ or C5′ position of the nucleoside. In the absence of a test compound that interacts with the nucleic acid target, hydrophobic interactions will likely lead to stacking of the fluorescent label(s) on top of the terminal base pair, leading to a first level of fluorescence (“low fluorescence”). Upon association of a test compound with the terminus of the nucleic acid target, fluorescence increases (relative to the stacked conformation) due to release of the fluorescent label(s) from the stacking conformation (as a result of the interaction of a test compound with the nucleic acid target at the labeled terminus, see FIG. 4). The change of fluorescence is indicative of the interaction of the test compounds with to the nucleic acid target. The degree of fluorescence increase correlates with the binding affinity and concentration of the binding ligand. For example, the higher the affinity, the greater the degree of fluorescence increase. Similarly, within certain concentration ranges, the higher the concentration, the greater the degree of fluorescence increase. Therefore, measurement of the fluorescence signal as a function of the test compound concentration will allow the quantitative determinination of the binding affinity.

In one embodiment of the present invention, the test compound is selected from the group consisting of cyclodextrin, cyclodextrin derivative, cyclodextrin-based copolymer, polyamine, poly-imine, lipid-based nanoparticle, peptide comprising basic amino acids, and the like, as well as combinations of any two or more thereof. Cyclodextrin or cyclodextrin derivatives may be in the form of α-cyclodextrin, β-cyclodextrin or γ-cyclodextrin. The peptide may comprise lysine, arginine, histidine, and combinations thereof.

Cyclodextrins (CDs), are a group of cyclic polysaccharides comprising six to eight naturally occurring D(±)-glucopyranose units in alpha-(1,4) linkage. The numbering of the carbon atoms of D(+)-glucopyranose units is illustrated below.

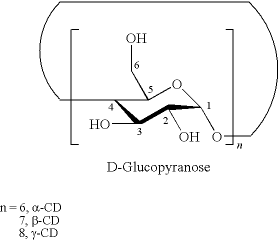

CDs are classified by the number of glucose units they contain: α-cyclodextrin has six glucose units; β-cyclodextrin has seven; and γ-cyclodextrin has eight. Each glucopyranose unit is referred to as ring A, ring B, etc., as exemplified below for β-CD.

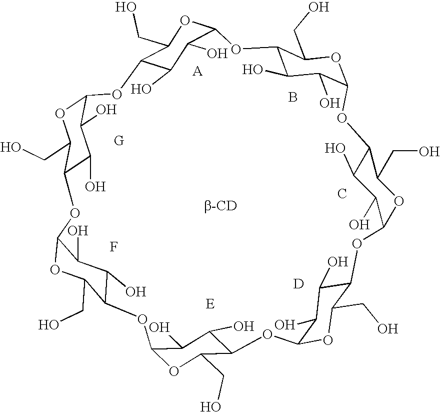

The three-dimensional architecture of CDs is unique in that they consist of cup-like shapes with relatively polar exteriors and nonpolar interiors. The unique amphiphilic structure is thought to be able to imbibe hydrophobic compounds to form host-guest complexes. According to both in vitro and in vivo studies, CDs, especially alkylated CD derivatives, may have enhancer activity on transport through cell membranes. For example, Agrawal et al. (U.S. Pat. No. 5,691,316) describes a composition including an oligonucleotide complexed with a CD to achieve enhancing cellular uptake of oligonucleotide.

In another embodiment, the test compound is represented by a construct of formula 1: CD¹-L¹-CD²-CA²(I),

wherein:

CD=cyclodextrin;

L¹, L²=linker; and

CA¹, CA²=cationic arm.

Each linker of the constructs may he independently selected from the group consisting of a covalent bond, a disulfide linkage, a protected disulfide linkage, an ether linkage, a thioether linkage, a sulfoxide linkage, an amine linkage, a hydrazone linkage, a sulfonamide linkage, an urea linkage, a sulfonate linkage, an ester linkage, an amide linkage, a carbamate linkage, a dithiocarbamate linkage, and the like, as well as combinations thereof. The linkers may be covalently linked to the 6-positions of A,D-rings, A,C-rings or A,E-rings of cyclodextrin.

Linkers with more than one orientation for attachment to cyclodextrin can be employed in all possible orientations for attachment. For example, an ester linkage may be orientated as —OC(O)— or —C(O)O—; a sulfonate linkage may be orientated —OS(O)₂— or —S(O)₂O—; a thiocarbamate linkage may be orientated —OC(S)NH— or —NHC(S)O—. A skilled artisan will readily recognize other suitable linkers for attachment of each positively charged arm.

In some embodiments, the cationic arms comprise a plurality of residues selected from amines, guanidines, amidines, N-containing heterocycles, or combinations thereof. In related embodiments, one or both of the cationic arms further comprises neutral and/or polar functional groups. In related embodiments, each cationic arm may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. In preferred embodiments, each cationic arm may be an oligomer selected from the group consisting of oligopeptide, oligoamide, cationically functionalized oligoether, cationically functionalized oligosaccharide, oligoamine, oligoethyleneimine, and the like as well as combinations thereof. The oligomers may be oligopeptides where all the amino acid residues of the oligopeptide are capable of forming positive charges. Yet in other embodiments, the length of the contiguous backbone of each cationic arm is about 12 to 200 Angstroms. For example, the cationic arms may be oligopeptides comprising 3 to 15 amino acids (approximately 12 to 80 Angstroms); preferably 3 to 10 amino acids (approximately 12 to 55 Angstroms).

As used herein, the term “amino acids” include the (D) and (L) stereoisomers of such amino acids when the structure of the amino acid admits stereoisomeric forms. The configuration of the amino acids and amino acid residues herein are designated by the appropriate symbols (D), (L) or (DL), furthermore when the configuration is not designated the amino acid or residue can have the configuration (D), (L) or (DL).

As used herein, the term “cationically functionalized oligosaccharide” is an oligosaccharide comprising one or more “cationically functionalized monosaccharides.”

As used herein, the term “cationically functionalized ethylene glycols” may include any substituted ethylene glycols where the substituents comprise functional groups that can form positive charge, e.g. amine and phosphorus containing groups.

As used herein, the term “cationically functionalized oligoether” may include any substituted oligoether where the substituents comprise functional groups that can form positive charge, e.g, amine and phosphorus containing groups,

In accordance with the present invention, the length of the contiguous backbone of the cationic arms is selected so as to correspond to the specific nucleic acid targets which are intended to interact with the molecular entities. In some embodiments, the length of the contiguous backbone of each of the cationic arms is 12 to 200 Angstroms; preferably 12 to 160 Angstroms; more preferably 12 to 120 Angstroms; most preferably 12 to 80 Angstroms. For example, when the CD core provides an anchor for one end of a nucleic acid strand, and assuming that the closest distance between two stacked nucleotides is around 2.5 Angstroms, the lower limit of 12 Angstroms for the arm length corresponds to a nucleic acid of about 5 nucleotides while the upper limit of 200 Angstroms corresponds to about 80 nucleotides.

Examples of constructs prepared utilizing beta-CD functionalized 6-amine linkage are illustrated in Scheme 1. Oligopeptides with positive charged functional groups can be readily prepared by standard peptide chemistry. Oligoamines can be readily prepared by known methods or are commercially available. The linkage between A⁶,D⁶-amine of CD and oligopeptides or oligoamines can readily be accomplished by amide bond formation.

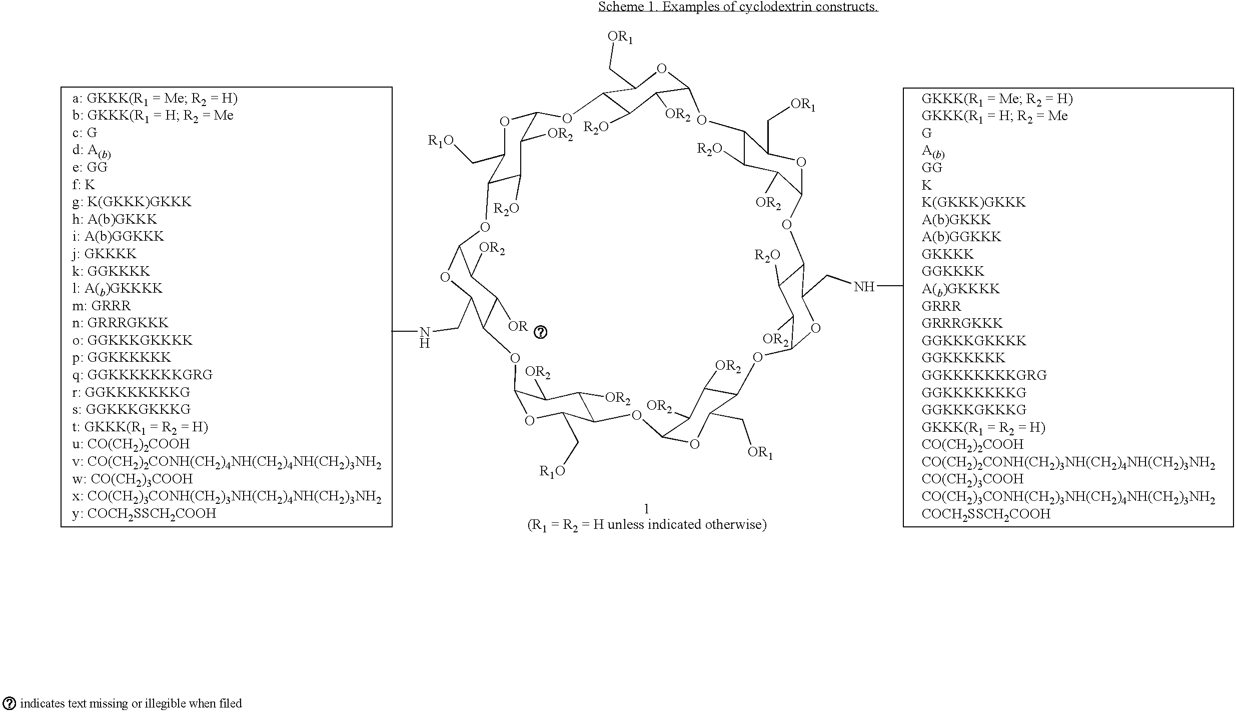

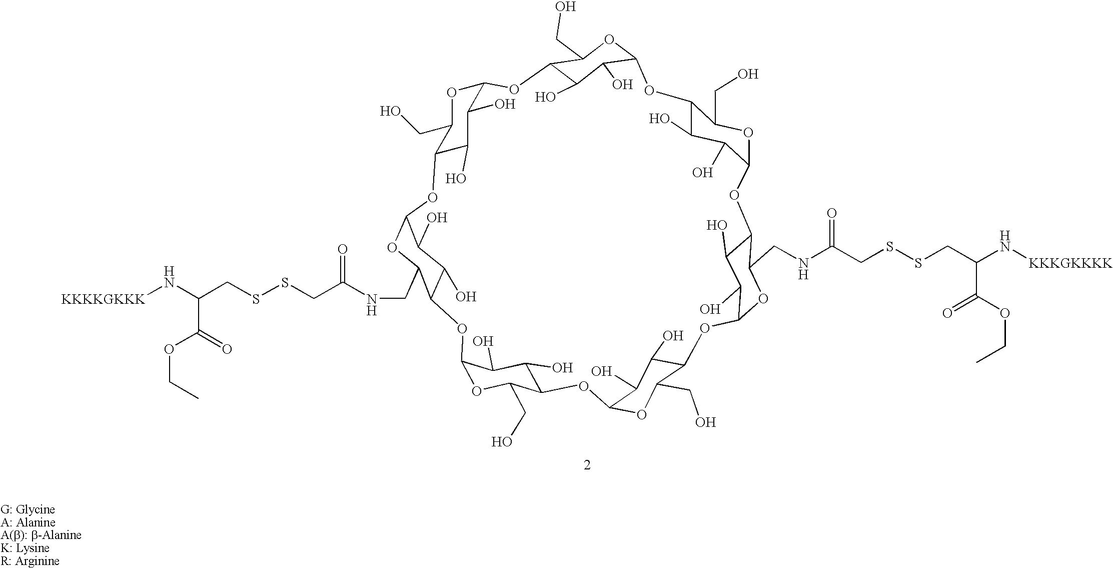

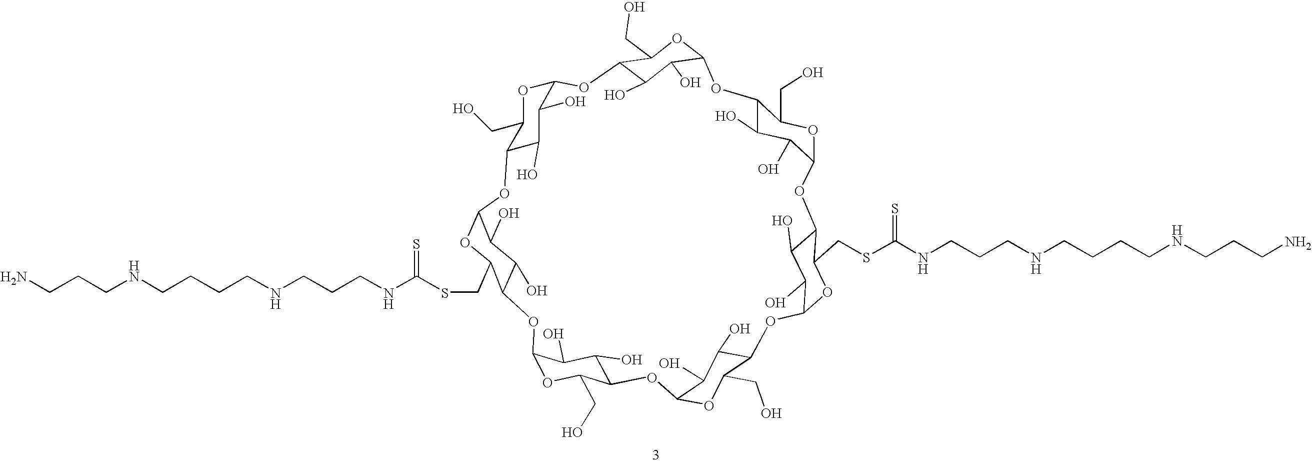

Each box in Scheme 1 discloses SEQ ID NOS 7, 7-19 and 7, respectively, in order of appearance. The sequences “KKKKGKKK” and “KKKGKKKK” are disclosed as SEQ ID NOS 20-21, respectively.

In yet another embodiment, the nucleic acid is double stranded nucleic acid with at least one blunt end or with at least one nucleotide overhang (e.g. siRNA).

As used herein, the term “nucleic acids” are oligonucleotides such as deoxyribonucleic acid (DNA) or ribonucleic acid (RNA), or chimeric oligonucleotides, containing DNA and RNA, or oligonucleotide strands containing non-natural monomers, including but not limited to 2′-methoxy or 2′-fluoro-modified nucleotides with ribo- or arahino- stereochemistry at the 2′-position, nucleotides comprising sugar mimetic parts, “acyclic” nucleotides or thio-substituted phosphate groups. Nucleic acids contemplated for use in the practice of the present invention may also include conjugated nucleic acids where nucleic acids conjugate to protein, polypeptide or any organic molecules.

As used herein, “acyclic nucleotides” refers to any nucleotide having an acyclic ribose sugar, or an acyclic ribose-sugar like structure, for example where any of the ribose carbons are independently or in combination absent from the nucleotide or disconnect from each other.

As used herein, “double-stranded nucleic acids (hybrids)” are formed from two individual oligonucleotide strands of substantially identical length and complete or near-complete sequence complementarity (“blunt end hybrids”) or offset sequence complementarity (“symmetrical overhang hybrids”, not necessarily implying sequence identity of the overhanging monomers), or from strands of different lengths and complete or offset sequence complementarity (“overhang hybrids”). In symmetrical overhang hybrids, the number of non-hybridized overhang nucleotides may be between 1-10.

As used herein, “sequence complementarity” is defined as the ability of monomers in two oligonucleotides to form base pairs between one nucleotide in one strand and another nucleotide in the second strand by formation of one or more hydrogen bonds between the monomers in the base pair.

As used herein, “complete sequence complementarity” means that each residue in a consecutive stretch of monomers in two oligonucleotides participates in base pair formation.

As used herein, “near-complete sequence complementarity” means that a consecutive stretch of base pairs is disrupted by no greater than one unpaired nucleotide per 3 consecutive monomers involved in base pairing. Preferably, base pairing refers to base pairs between monomers that follow the Watson-Crick rule (adenine-thymine, A-T; adenine-uracil, A-U; guanine-cytosine, G-C) or form a wobble pair (guanine-uracil,

As used herein, “hairpin nucleic acids” are funned from a single oligonucleotide strand that has complete or near-complete sequence complementarity or offset sequence complementarity between stretches of monomers within the 5′ and 3′ region such that, upon formation of intra-oligonucleotide base pairs, a hairpin structure is formed that consists of a double-stranded (hybridized) domain and a loop domain which contains nucleotides that do not participate in pairing according to the Watson-Crick rule. Preferred length of hairpin oligonucleotides is between 15-70 monomers (nucleotides); more preferred length is between 18-55 monomers; even more preferred length is between 20-35 monomers; most preferred length is between 21-23 monomers. A skilled artisan will realize nucleotides at the extreme 5′ and 3′ termini of the hairpin may but do not have to participate in base pairing.

The teens “polynucleotide” and “nucleic acid molecule” are used broadly herein to refer to a sequence of two or more deoxyribonucleotides, ribonucleotides or analogs thereof that are linked together by a phosphodiester bond or other known linkages. As such, the terms include RNA and DNA, which can be a gene or a portion thereof, a cDNA, a synthetic polydeoxyribonucleic acid sequence, or the like, and can he single stranded or double stranded, as well as a DNA/RNA hybrid. The terms also are used herein to include naturally occurring nucleic acid molecules, which can be isolated from a cell using recombinant DNA methods, as well as synthetic molecules, which can be prepared, for example, by methods of chemical synthesis or by enzymatic methods such as by PCR. The term “recombinant” is used herein to refer to a nucleic acid molecule that is manipulated outside of a cell, including, for example, a polynucleotide encoding an siRNA specific for a histone H4 gene operatively linked to a promoter. Preferred length of oligonucleotides in double-stranded nucleic acids is between 15-60 monomers; more preferred length is between 15-45 monomers; even more preferred length is between 19-30 monomers; most preferred length is between 21-27 monomers.

In yet another embodiment, the fluorescent label(s) in the methods comprise fluorescent nucleobase analogue(s) that replace nucleobase(s) at one or both of terminus nucleotide(s). The fluorescent nucleobase analogue(s) may be 2-aminopurine (2AP), 2,6-diaminopurine, formycin, 4-amino-6-methyl-pteridone, etheno-A, 3-methylisoxanthopterin (3MI), 6-methylisoxanthopterin (6MI), isoxanthopterin, pyrrole-(d)C, 5-(1-pyrenylethynyl)-(d)C, furano-(d)T, isoxanthine, 5-(1-pyrenylethynyl)-U, benzo-U, lumazine, or the like.

In yet another embodiment, the fluorescent label may be attached to a nucleoside at C2′, C3′, C4′ or C5′ position of said nucleoside via a linker. Under this condition, the fluorescent label may he a pyrene, a fluorescein, a coumarin, an Alexa floors, a BODIPY, a xanthene, a naphthylamine, a fluorescein, a rhodamine, a cyanine dye comprising Cy3 or Cy5, a fluorescein derivative (e.g. tetrachloro-fluorescein), a TAMRA, or the like; preferably a pyrene. The linker is a linear chain of C₂-C₂₀ alkyl, or —(X(CH₂)ₘ)ₙ— wherein X is independently O, S, NH, C═O, O—C—O or NHC═O, m=1 -5 and n=1-7; preferably X═O, n=2 and n=1-3.

In some embodiments, the present invention provides compositions that comprise nucleic acid having fluorescent label(s) attached at one or both termini thereof via a linker wherein said linker is a linear chain of C₂-C₂₉ alkyl, or —(X(CH₂)ₘ)ₙ— wherein X is independently O, S, NH, C═O, O—C═O or NHC═O, m=1-5 and n=1-7; preferably X═O, m=2, and n=1-3. For example, the linker may be —OCH₂CH₂CH₂—, —NHC═O—CH₂CH₂CH₂— or OCH₂CH₂CH₂—NHC═O—CH₂CH₂CH₂—.

In yet another embodiment, the present invention provides assay kits for screening for compounds that bind a nucleic acid target at one or both termini thereof, comprising a nucleic acid modified by the incorporation of fluorescent label(s) at one or both termini thereof. The assay kits further comprises one or more test compounds selected from the group consisting of cyclodextrin, cyclodextrin derivative, cyclodextrin-based copolymer, polyamine, poly-imine, lipid-based nanoparticle, peptide comprising basic amino acids, and the like, as well as combinations of any two or more thereof. Cyclodextrin or cyclodextrin derivative may be in a form of α-cyclodextrin, β-cyclodextrin or γ-cyclodextrin. The peptide may comprise lysine, arginine, histidine, and combinations thereof. In another embodiment, the test compound is represented by a construct of formula I.

In yet another embodiment, the nucleic acid is double stranded nucleic acid with at least one blunt end or with at least one nucleotide overhang. In yet anther embodiment, the fluorescent label(s) in the methods comprise fluorescent nucleobase analogue(s) that replace nucleobase(s) at one or both of terminus nucleotide(s). The fluorescent nucleobase analogue(s) may be 2-aminopurine (2AP), 2,6-diaminopurine, formycin, 4-amino-6-methyl-pteridone, etheno-A, 3-methylisoxanthopterin (3MI), 6-methylisoxanthopterin (6MI), isoxanthopterin, pyrrole-(d)C, 5-(1-pyrenylethynyl)-(d)C, furano-(d)T, isoxanthine, 5-(1-pyrenylethynyl)-U, benzo-U, lumazine, or the like. In yet another embodiment, the fluorescent label may be attached to a nucleoside at C2′, C3′, C4′ or C5′ position of said nucleoside via a linker. Under this condition, the fluorescent label may be a pyrene, a fluorescein, a coumarin, an Alexa fluors, a BODIPY, a xanthene, a naphthylamine, a fluorescein, a rhodamine, a cyanine dye comprising Cy3 or Cy5, a fluorescein derivative (e.g. tetrachloro-fluorescein), a TAMRA, or the like; preferably a pyrene. The linker is a linear chain of C₂-C₂₀ alkyl, or —(X(CH₂)ₘ)ₙ— wherein X is independently O, S or NH, m=1 -5 and n=1-7; preferably X═O, n=2 and n=1-3. The assay kits may optionally further comprise means for determining the fluorescence of the modified nucleic acids, and means for comparing the result of said determining to the result of said measuring to ascertain any difference in fluorescence.

### Examples

The invention now being generally described, it will be more readily understood by reference to the following examples, which are included merely for purposes of illustration of certain aspects and embodiments of the present invention, and are not intended to limit the invention.

Fluorescence measurements were performed on a thermostatted RF-5301PC spectrofluorometer at 25° C. Fluorescent spectra were recorded in 10-50 mM sodium cacodylate buffer, pH 6.5, or 10-50 mM HEPES (4-(2-hydroxyethyl)-1-piperazineethanesulfonic acid) buffer, pH 7.0, at specified RNA concentration while irradiating at a wavelength of 310 nm for 2AP or 340 nm for pyrene fluorescent labels.

### Example 1

### Binding Assay with Pyrene-Labeled RNA

A pyrene labeled nucleic acid target was employed to screen test compounds that may interact with the terminus of the nucleic acid target. A double-stranded RNA labeled on the 3′ strand with a fluorescent pyrene via an amino-butyryl linker was used as the nucleic acid target and obtained from commercial chemical custom synthesis. The RNA construct (SEQ ID NO: 1) contains a single uridine to 2′-amino-butyryl-pyrene uridine substitution as shown below.

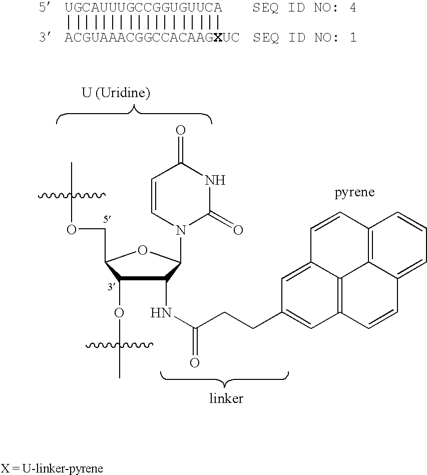

The RNA was at 100 nM concentration in aqueous buffer. Potential RNA binders (test compounds) were screening against the pyrene-labeled target RNA construct. Increasing amounts of compound were added and the fluorescence signals of pyrene against baseline were recorded. The binding affinity of the test compounds were determined by fitting a sigmoidal dose response curve and calculating the half point of the signal change (EC50).

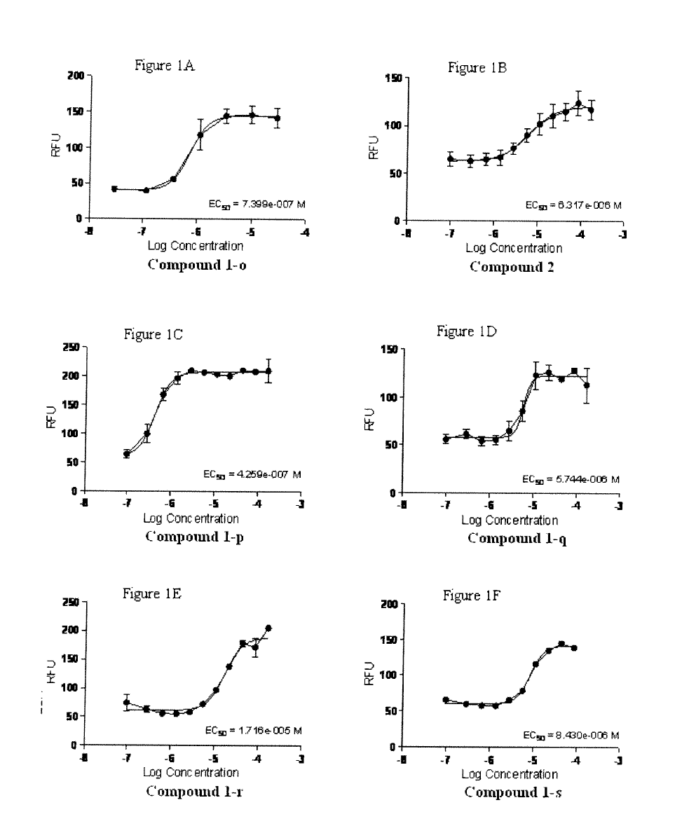

The results of the assays are shown in FIGS. 1A-1F. The intensity of the flurescence signal (at the emission wavelength of 340 nm) was plotted over the concentration of the added compound. The increase of fluorescence, shown as a sigmoidal dose-response curve, indicates release of the fluorescent label, e.g. pyrene, as a result of interaction of the test compound with the nucleic acid target at the labeled terminus (see FIG. 4). Among many screened test compounds, several exemplary compounds identified to have desirable binding affinities against the pyrene-labeled target RNA construct (SEQ ID NO: 1) include compounds 1-o, 2, 1-p, 1-q, 1-r and 1-s; see Scheme 1. These results demonstrate that a feasible method according to the invention for screening compounds for the ability to interact with a nucleic acid target where the nucleic acid target has been modified by the incorporation of fluorescent label, such as pyrene, via linker at one terminus of the target.

### Example 2

### Binding Assay with 2AP-Labeled RNA

A 2-aminopurine (2AP) labeled nucleic acid target was employed to screen test compounds that may interact with the terminus of the nucleic acid target. Double-stranded RNAs (SEQ ID NOs 2 and 3) were used as nucleic acid targets and obtained from commercial chemical custom synthesis in which one terminal base pair involved a fluorescent 2-aminopurine (2AP).

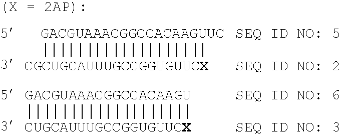

The RNA constructs were at 100 nM concentration in aqueous buffer. Potential RNA binders (test compounds) were screened against the 2AP-labeled target RNA constructs. Increasing amounts of test compound were added and the fluorescence signals of 2AP were recorded. The binding affinity was determined by fitting a sigmoidal dose response curve and calculating the half point of the signal change (EC50).

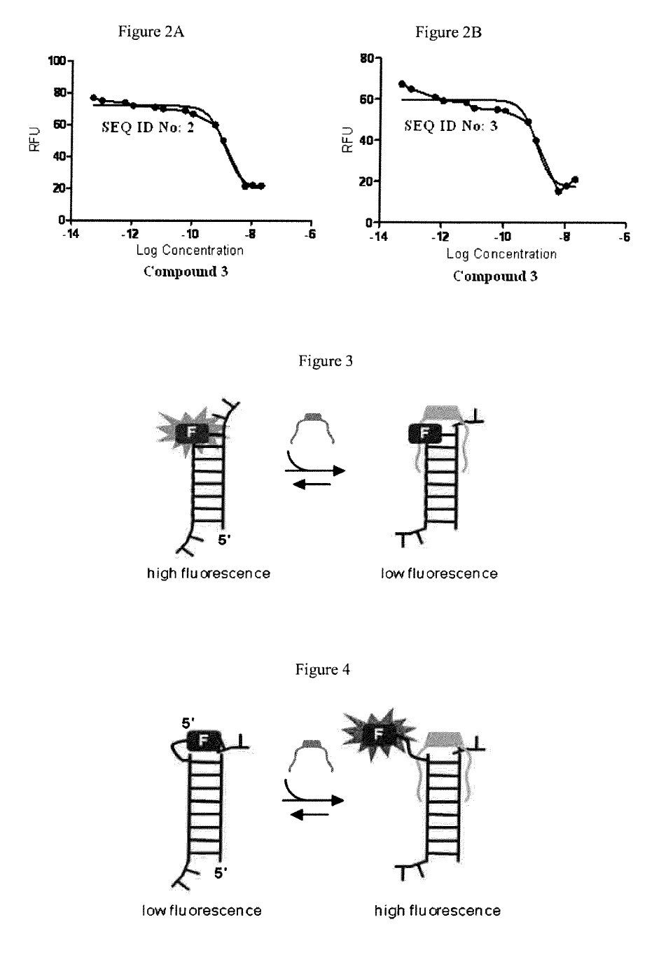

The results are shown in FIGS. 2A and 2B. The intensity of the flurescence signal (at the emission wavelength of 310 nm) was plotted over the concentration of the added compound. The decrease of fluorescence, shown as a sigmoidal dose-response curve, indicates quenching of the fluorescent label, e.g. 2AP, as a result of interaction of the target nucleic acid with the binding test compound (see FIG. 3). Among many tested compounds, the exemplary compound identified to have desirable binding affinities against various 2AP-labeled target RNA constructs (SEQ ID NO: 2 and SEQ ID NO: 3) includes compound 3; see Scheme 1. These results demonstrate that a feasible method according to the invention for screening compounds for the ability to interact with a nucleic acid target where the nucleic acid target has been modified by the incorporation of fluorescent label, such as 2AP at one terminus of the target.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and arc not intended as limitations on the scope. Changes therein and other uses will occur to those skilled in the art, which are encompassed within the spirit of the invention, are defined by the scope of the claims.

Definitions provided herein are not intended to be limiting from the meaning commonly understood by one of skill in the art unless indicated otherwise.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications arc possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method for screening compounds for the ability to interact with a nucleic acid target comprising measuring the fluorescence of the nucleic acid target after contacting said nucleic acid target with a test compound, wherein said nucleic acid target has been modified by the incorporation of fluorescent label(s) at one or both termini thereof.

2. The method of claim 1, wherein said test compound is selected from the group consisting of cyclodextrin, cyclodextrin derivative, cyclodextrin-based copolymer, polyamine, lipid-based nanoparticle, peptide comprising basic amino acid units, poly-imine, and combination thereof.

3. The method of claim 2, wherein said test compound is represented by a construct of formula I:
CA¹-L¹-CD-L²-CA² (1)
wherein,
CD=cyclodextrin;
L¹, L²=linker; and
CA¹, CA²=cationic arm.

4. The method of claim 1, wherein said nucleic acid is double stranded nucleic acid with at least one blunt end.

5. The method of claim 1, wherein said nucleic acid is double stranded nucleic acid with at least one nucleotide overhang.

6. The method of claim 1, wherein said fluorescent label(s) comprise fluorescent nucleobase analogue(s) that replace nucleobase(s) at one or both of terminus nucleotide(s).

7. The method of claim 6, wherein said fluorescent nucleobase analogue(s) are 2-aminopurine (2AP), 2,6-diaminopurine, formycin, 4-amino-6-methyl-pteridone, etheno-A, 3-methylisoxanthopterin (3MI), 6-methylisoxanthopterin (6MI), isoxanthopterin, pyrrole-(d)C, 5-(1-pyrenylethynyl)-(d)C, furano-(d)T, isoxanthine, 5-(1-pyrenylethynyl)-U, benzo-U or lumazine.

8. The method claim 1, wherein said fluorescent label is attached to a nucleoside at C2′, C3′, C4′ or C5′ position of said nucleoside via a linker.

9. The method of claim 9, wherein said fluorescent label is a pyrene, a fluorescein, a coumarin, an Alexa fluors, a BODIPY, a xanthene, a naphthylamine, a fluorescein, a rhodamine, a cyanine dye, a fluorescein derivative, or a TAMRA.

10. The method of claim 9, wherein said linker is a linear chain of C₂-C₂₀ alkyl, or —(X(CH₂)ₘ)ₙ— wherein X is independently O, S, NH, C═O, O—C═O or NHC═O, m=1 -5 and n=1-7.

11. A composition comprising nucleic acid having fluorescent label(s) attached at one or both termini thereof via a linker wherein said linker is a linear chain of C2-C₂₀ alkyl, or —(X(CH₂)ₘ)ₙ— wherein X is independently O, S, NH, C═O, O—C═O or NHC═O, m=1-5 and n=1-7.

12. An assay kit, for screening for compounds that bind a nucleic acid target at one or both termini thereof, said kit comprising;
a nucleic acid modified by the incorporation of fluorescent labels) at one or both termini thereof, and
one or more test compounds selected from the group consisting of cyclodextrin, cyclodextrin derivative, cyclodextrin-based copolymer, polyamine, lipid-based nanoparticle, peptide comprising basic amino acid units and poly-imine.

13. The assay kit of claim 12, wherein said assay kit comprises one or more test compounds represented by formula I:
CA¹-L¹-CD-L²-CA² (I)
wherein,
CD=cyclodextrin;
L¹, L²=linker; and
CA¹, CA²=cationic arm.

14. The assay kit of claim 12, wherein said nucleic acid target is double stranded nucleic acid with at least one blunt end.

15. The assay kit of claim 12, wherein said nucleic acid target is double stranded nucleic acid with at least one nucleotide overhang.

16. The assay kit of claim 12, wherein said fluorescent label(s) comprise fluorescent nucleobase analogue(s) that replace the corresponding nucleotide(s) of said nucleic acid at one or both termini thereof.

17. The assay kit of claim 16, wherein said fluorescent nucleobase analogue(s) are 2-aminopurine (2AP), 2,6-diaminopurine, formycin, 4-amino-6-methyl-pteridone, etheno-A, 3-methylisoxanthopterin (3MI), 6-methylisoxanthopterin (6MI), isoxanthopterin, pyrrole-(d)C, 5-(1-pyrenylethynyl)-(d)C, furano-(d)T, isoxanthine, 5-(1-pyrenylethynyl)-U, benzo-U or lumazine.

18. The assay kit of claim 12, wherein said fluorescent label is attached to a nucleoside at C2′, C3′, C4′ or C5' position of said nucleoside via a linker.

19. The assay kit of claim 18, wherein said fluorescent label is a pyrene, a fluorescein, a coumarin, an Alexa floors, a BODIPY, a xanthene, a naphthylamine, a fluorescein, a rhodamine, a cyanine dye, a fluorescein derivative, or a TAMRA.

20. The assay kit of claim 12, further comprising:
means for determining the fluorescence of the modified nucleic acids, and
means for comparing the result of said determining to the result of said measuring to ascertain any difference in fluorescence.
